(19) United States
(12) Patent Application Publication
(10) Pub. No.: US 2008/0167325 A1
(43) Pub. Date: Jul. 10, 2008

(54) VALACYCLOVIR COMPOSITIONS

(76) Inventors: Praveen Kumar BS, Coorg (IN); Rahul Sudhakar Gawande, Nagpur (IN); Ravinder Kodipyaka, Kaghaznagar (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/963,946

(22) Filed: Dec. 24, 2007

Related U.S. Application Data
(60) Provisional application No. 60/894,714, filed on Mar. 14, 2007.

(30) Foreign Application Priority Data
Dec. 27, 2006 (IN) 2430/CHE/2006

(51) Int. Cl. A61K 31/522; C07D 473/30; A61P 31/12

(52) U.S. Cl. 514/263.3; 544/265

(57) ABSTRACT

Solid pharmaceutical compositions of valacyclovir or pharmaceutically acceptable salts thereof. Embodiments of the invention relate to processes for preparing solid pharmaceutical compositions, avoiding a wet granulation step.

### INTRODUCTION TO THE INVENTION

The present invention relates to solid pharmaceutical compositions of valacyclovir or pharmaceutically acceptable derivatives thereof.

The present invention also relates to process of preparing solid pharmaceutical compositions of valacyclovir or pharmaceutically acceptable derivatives thereof.

The present invention relates to processes, which avoid a wet granulation step and produce stable solid pharmaceutical compositions.

Valacyclovir is the L-valine ester of the antiviral drug acyclovir, and is a prodrug of acyclovir. Valacyclovir has a chemical name L-valine, 2-[(2-amino-1,6-dihydro-6-oxo-9H-purin-9-yl)methoxy]ethyl ester, with a molecular formula C₁₃H₂₀N₆O₄, molecular weight 324.336 g/mol, and structural Formula I.

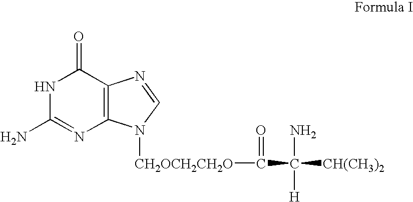

The hydrochloric acid salt, valacyclovir hydrochloride, is a white to off-white powder. It is a crystalline solid, occurring as a hydrate. The maximum solubility in water at 25° C. is 174 mg/mL. The pKa values for valacyclovir hydrochloride are 1.90, 7.47 and 9.43.

The compound 2-amino-1,9-dihydro-9-[(2-hydroxyethoxy)methyl]-6H-purin-6-one, also known as acyclovir, possesses potent antiviral activity and is widely used in the treatment and prophylaxis of viral infections in humans, particularly infections caused by herpes group viruses in humans. However, acyclovir is poorly absorbed from the gastrointestinal tract upon oral administration and this low bioavailability means that multiple high doses orally may need to be administered, especially for the treatment of less sensitive viruses or infections in order to achieve and maintain effective anti-viral levels in the plasma.

The L-valine ester of acyclovir, i.e., valacyclovir, has been shown to possess much improved bioavailability while retaining the anti-viral properties of acyclovir.

Valacyclovir is rapidly and almost entirely (˜99%) converted to the active compound, acyclovir, and L-valine by first-pass hepatic metabolism through enzymatic hydrolysis. Neither valacyclovir nor acyclovir is metabolized by cytochrome P450 enzymes.

Valacyclovir is phosphorylated by viral thymidine kinase to acyclovir triphosphate (the active metabolite), which then inhibits herpes viral DNA replication by competitive inhibition of viral DNA polymerase, and by incorporation into and termination of the growing viral DNA chain. When used as a substrate for viral DNA polymerase, acyclovir triphosphate competitively inhibits dATP leading to the formation of ‘faulty’ DNA. This is where acyclovir triphosphate is incorporated into the DNA strand replacing many of the adenosine bases. This results in the prevention of DNA synthesis, as phosphodiester bridges can longer to be built, destabilizing the strand.

Valacyclovir hydrochloride is currently marketed as VALTREX® by GlaxoSmithKline. VALTREX® caplets are intended for oral administration and made available in two strengths, 500 mg and 1 gram. Each caplet contains valacyclovir hydrochloride equivalent to 500 mg or 1 gram of valacyclovir and the inactive ingredients carnauba wax, colloidal silicon dioxide, crospovidone, FD&C Blue No. 2 lake, hypromellose, magnesium stearate, microcrystalline cellulose, polyethylene glycol, polysorbate 80, povidone and titanium dioxide. The blue, film-coated caplets are printed with edible white ink.

Approved therapeutic indications for VALTREX® include the following:

1) Treatment of herpes zoster (shingles).

2) Treatment or suppression of genital herpes in immuno competent individuals and for the suppression of recurrent genital herpes in HIV-infected individuals.

3) Treatment of cold sores.

Valacyclovir is disclosed in U.S. Pat. No. 4,957,924. U.S. Pat. No. 6,107,302 discloses an anhydrous crystalline form of valacyclovir. U.S. Patent Application Publication Nos. 2003/0114470, 2004/0197396, and 2005/0043329, and International Application Publication Nos. WO 2004/052892, WO 2004/106338, and WO 2005/000850 disclose crystalline forms of valacyclovir hydrochloride.

U.S. Pat. No. 5,879,706 and International Application Publication Nos. WO 2004/000265, WO 2001/82905, and WO 1997/025989 disclose pharmaceutical compositions of valacyclovir hydrochloride.

Valacyclovir has proven to be difficult to formulate. In the above documents it has been shown that it is difficult to obtain a tablet of sufficient hardness and friability for pharmaceutical handling and for film coating. One problem is that valacyclovir has “adhesive properties” that cause sticking of powder blends to tablet forming dies and therefore needs to be efficiently lubricated. The formulation of granules of valacyclovir was found to be problematic due to the adhesive properties of valacyclovir and also to problems relating to the pH dependent dissolution rate and discoloration of the granules. Granules of valacyclovir itself tend to be fragile and have a low coating efficacy.

However, the aforementioned documents disclose use of granulation steps or use of solvents for the processes of preparing pharmaceutical compositions. Use of granulation steps always requires means to remove the granulation liquid at a later stage in order to arrive at the final solid compositions. Use of solvents such as water may give rise to reduced stability. Use of non aqueous solvents may give rise to unwanted impurities or residual solvents which are always desired to be absent in the pharmaceutical compositions. Use of solvents may also lead to undesired polymorphic changes of active ingredient.

Surprisingly it has been found that when valacyclovir hydrochloride formulations are processed according to the present invention, the processing is simple and all physical parameters are found to be satisfactory.

### SUMMARY OF THE INVENTION

The present invention relates to solid pharmaceutical compositions of valacyclovir or pharmaceutically acceptable salts thereof.

The present invention also relates to processes for preparing solid pharmaceutical compositions of valacyclovir or pharmaceutically acceptable salts thereof.

The present invention relates to processes, which avoid a wet granulation step and produce stable solid pharmaceutical compositions.

In an embodiment the present invention includes processes comprising steps such as direct compression or dry granulation.

An embodiment provides processes for preparing solid pharmaceutical compositions of valacyclovir or a salt thereof, comprising:

(i) Dry mixing of valacyclovir or a salt thereof with at least one pharmaceutical excipient.

(ii) Dry processing of the mixture obtained in step (i) into a desired solid form.

In an embodiment the present invention includes use of valacyclovir hydrochloride having particle size distributions, wherein D₉₀ is within the range of about 350 μm to about 500 μm.

In an embodiment the present invention relates to methods of using the pharmaceutical compositions of valacyclovir or its salts.

### DETAILED DESCRIPTION

The present invention relates to solid pharmaceutical compositions of valacyclovir or pharmaceutically acceptable salts thereof.

The present invention also relates to process of preparing solid pharmaceutical compositions of valacyclovir or pharmaceutically acceptable salts thereof.

The present invention relates to processes, which avoid a wet granulation step to produce stable solid pharmaceutical compositions.

In an embodiment the present invention includes process steps such as direct compression or dry granulation techniques.

In another embodiment processes according to the invention for preparing a solid pharmaceutical composition of valacyclovir or a salt thereof comprise:

(i) Dry mixing of valacyclovir or a salt thereof with at least one pharmaceutical excipient.

(ii) Dry processing of the mixture obtained in step (i) to produce the desired solid form.

In an embodiment the present invention relates to methods of using the pharmaceutical compositions of valacyclovir or its salts.

A pharmaceutically acceptable derivative includes any pharmaceutically acceptable ester or salt of valacyclovir, including any polymorphic forms thereof, which, upon administration to a patient, is capable of providing (directly or indirectly) valacyclovir or an antivirally active metabolite or residue thereof.

The pharmaceutically acceptable salts of valacyclovir can be acid addition salts derived from an appropriate acid. The acids that are widely used include but not limited to hydrochloric, sulphuric, phosphoric, maleic, fumaric, citric, tartaric, lactic, acetic or p-toluenesulphonic acids. Commonly used salts include the hydrochloride salt of valacyclovir.

Valacyclovir is rapidly converted to acyclovir, which has demonstrated antiviral activity against herpes simplex virus types I (HSV-1) and 2 (HSV-2) and varicella-zoster virus (VZV) both in vitro and in vivo.

The antiviral activity of acyclovir is highly selective due to its affinity for the enzyme thymidine kinase (TK) encoded by HSV and VZV. This viral enzyme converts acyclovir into acyclovir monophosphate, a nucleoside analogue. The monophosphate is further converted into a diphosphate by cellular guanylate kinase and into a triphosphate by a number of cellular enzymes. In vitro, acyclovir triphosphate stops replication of herpes viral DNA. This is accomplished in 3 ways: 1) competitive inhibition of viral DNA polymerase; 2) incorporation and termination of the growing viral DNA chain; and 3) inactivation of the viral DNA polymerase. The greater antiviral activity of acyclovir against HSV compared to VZV is due to its more efficient phosphorylation by the viral TK.

Valacyclovir can be unstable in presence of solvents as these may lead to polymorphic changes of the active ingredient or may degrade and form unwanted degradation products.

Valacyclovir is prone to degradation, giving rise to impurities. Some of the impurities include:

1) “Guanine,” 2-amino-1H-purin-6(9H)-one and represented by Formula II.

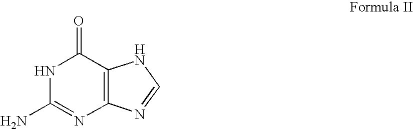

2) Acyclovir,” 2-amino-1,9-dihydro-9-[(2-hydroxyethoxy)methyl]-6H-purin-6-one and represented by Formula III.

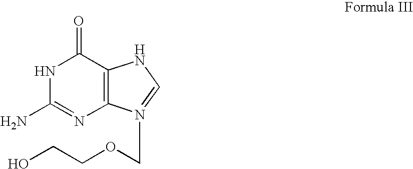

3) “Carbonyl,” 2-[(2-amino-6-oxo-1,6-dihydro-9H-purin-9-yl)methoxyl]ethyl N-[(benzyloxy)carbonyl]-L-valinate and represented by Formula IV.

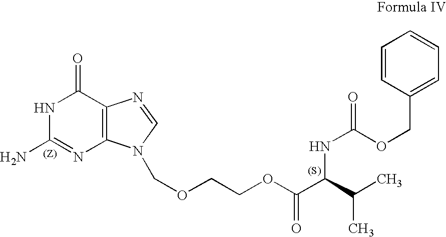

In one of the embodiments the invention includes stable pharmaceutical compositions comprising valacyclovir and at least one pharmaceutical excipient.

In an embodiment, the guanine impurity is present in the composition at not more than about 0.25% by weight of the concentration of valacyclovir or its salt.

In another embodiment the acyclovir impurity is present in the composition at not more than about 1% by weight of the concentration of valacyclovir or its salt.

In yet another embodiment the carbonyl impurity is present in the composition at not more than about 0.25% by weight of the concentration of valacyclovir or its salt.

In certain embodiments the amount of total impurities present in the composition is not more than about 2% by weight of the concentration of valacyclovir or its salt.

Total impurities for purposes of the present invention include anything other than valacyclovir, giving a peak in a high performance liquid chromatography (HPLC) chromatogram when a sample of valacyclovir or a salt thereof is analyzed.

The acyclovir, guanine and carbonyl impurity content in valacyclovir or a salt thereof can be analyzed, using a HPLC procedure such as that described below.

### Buffer Preparation:

1.54 grams of ammonium acetate is transferred into a 1000 ml volumetric flask, and the volume is diluted with Milli-Q water and mixed well. pH is adjusted to 5.5±0.05 with orthophosphoric acid.

### Mobile Phase A:

Mix buffer and acetonitrile in the ratio of 97:3 v/v respectively. Filter through a 0.45 μm Pall nylon 66 membrane filter and degas in a sonicator for about 10 minutes.

### Mobile Phase B:

Mix Milli-Q water and acetonitrile in a ratio of 30:70 v/v respectively and degas in a sonicator for about 10 minutes.

With the above mobile phases, a gradient program of 0.01, 18, 30, 55, 60, and 65 minutes is performed with varying compositions of mobile phase for each time period, as given in the table below:

| Time | | |
|---|---|---|
| (minutes) | % of Mobile Phase A | % of Mobile Phase B |
| 0.01 | 100 | 0 |
| 18 | 100 | 0 |
| 30 | 60 | 40 |
| 55 | 10 | 90 |
| 60 | 100 | 0 |
| 65 | 100 | 0 |

### Diluents:

Diluent 1: Purified (Milli-Q) water and ethanol 90:10 v/v, respectively.
Diluent 2: 3.4 grams of potassium hydrogen phosphate is taken in a 1000 ml volumetric flask and volume is made up with Milli-Q water and mixed well.

### Sample Preparation:

1) 20 tablets are weighed and crushed into a fine powder with a mortar and pestle.

2) Powder equivalent to 69 mg valacyclovir is weighed accurately into a 25 ml volumetric flask.

3) 15 ml of Diluent 1 is added to step 2 and the flask is shaken thoroughly to disperse the material completely and then is sonicated for 20 minutes with intermediate shaking, and finally the volume is made up with Diluent 1 and mixed well.

4) 10 ml of the above solution is centrifuged in a centrifuge tube with a cap at 4000 rpm for 10 minutes.

5) 2.0 ml of step 4 is transferred by pipette into a 10 ml volumetric flask, diluted to volume with Diluent 2 and mixed well.

### Chromatographic System:

Column: 250 mm×4.6 mm, Intertsil ODS-3V, 5 μm column or equivalent.

Flow rate: 1.0 mL/minute.

| Impurity | LOD | LOQ | RRT |
|---|---|---|---|
| Acyclovir | 0.003 | 0.003 | 0.25 |
| Gaunine | 0.002 | 0.002 | 0.44 |
| Carbonyl | 0.005 | 0.004 | 1.61 |

LOD = Limit of detection, % w/w.
LOQ = Limit of quantification, % w/w.
RRT = Relative retention time, when valacyclovir is assigned a value of 1.

In an embodiment the present invention includes pharmaceutical compositions comprising valacyclovir or its pharmaceutically acceptable derivatives in concentrations about 1% to about 95%, or about 25% to about 90%, or about 50% to about 85%, by weight of total composition.

As mentioned above, embodiments of processes to prepare valacyclovir compositions of the present invention avoid wet granulation. In an embodiment, a process includes steps of dry granulation or direct compression.

Dry granulation can be used when materials of a formulation have sufficient inherent binding or cohesive properties to form granules. Dry granulation refers to the process of granulating without the use of liquids. In order for a material to be dry granulated at least one of its constituents, either the active ingredient or a diluent must have cohesive properties.

A process known as “slugging” can perform dry granulation. In “slugging” the material to be granulated is first made into a large compressed mass or “slug,” typically using a tablet press with large flat-faced tooling. Allowing sufficient time for the air to escape from the material to be compacted may form a fairly dense slug. Compressed slugs are then comminuted through a desired mesh screen manually or automatically as, for example, by way of a comminuting mil. Formation of granules by “slugging” is also known as “precompression.” When tablets are made from the granulated slugged material, the process is referred to as the “double compression method.”

Dry granulation may also be performed using a “roller compactor”. In a roller compactor material particles are consolidated and densified by passing the material between two high-pressure rollers. The densified material from a roller compactor is then reduced to a uniform granule size by milling the uniform granules may then be mixed with other substances, such as a lubricant, to tablet the material (as, for example, by way of a rotary tableting machine).

Dry granulation has several advantages over wet granulation, including its usefulness with respect to ingredients that are sensitive to moisture or unable to withstand elevated temperatures during drying, and because it does not use organic solvents which may pose health and environmental hazards. There are also fewer steps involved in dry granulation than wet granulation. Dry granulation by means of roller compaction is an efficient and useful method of granulation capable of handing a large amount of material in a short period of time.

Direct compression is a useful method when the active ingredient shows inherently good compression behaviour. Tablet manufacturers can take advantage of reduced labour, easier processing and good end-product performance. The economic attractiveness of direct compression versus wet granulation tabletting becomes obvious when the process steps of the different technologies are compared. Wet granulation consists of weighing, preparation and addition of binder solution, screening, drying and blending of lubricants before tableting. Direct compression, on the other hand, consists of only three major steps: weighing, blending and tableting.

Direct compression is considered to be a very simple and cost-effective process for manufacturing pharmaceutical tablets.

Various parameters impacting compression processes are compactability, flow, particle size of the active ingredient and other properties of the excipients such as moisture content (determined by a Karl Fischer (KF) apparatus or infrared moisture balance), particle size (determined by sieve analyzer or Malvern particle size analyzer), bulk density and tapped density (such as is determined by USP density apparatus apparatus, flow properties (determined by Flowdex apparatus), compressibility, etc.

Particle size also can affect how freely crystals or a powdered form of a drug will flow, which has consequences in the production processing of pharmaceutical products containing the drug.

The percentages of particles with different dimensions that exist in a powder is called the particle size distribution. It is represented in certain ways. Particle size is the maximum dimension of a particle, normally expressed in units of μm. Particle size distributions can be expressed in terms of, D₁₀, D₅₀, D₉₀ and D_{[4,3]}. The D₁₀, D₅₀ and D₉₀ represent the 10th percentile, median or the 50th percentile, and the 90th percentile of the particle size distribution, respectively, as measured by volume. That is, the D₁₀, D₅₀, D₉₀ is are values of the distribution such that 10%, 50%, 90% by volume of the particles have sizes of these values or less, or is the percentage of particles smaller than those sizes. D₅₀ is also known as median diameter of particles. It is one of the important parameters representing characteristics of particle of powder. For a sample, if D₅₀=5 μm, it means that 50% of the particles are less than or equal to 5 μm. Similarly, if D₁₀=5 μm, 10% by volume of the particles are less than or equal to 5 μm, and if D₉₀=5 μm, 90% of the particles are less than or equal to 5 μm. D_{[4,3]} is the volume moment mean of the particles or the volume weighted particle size.

Particles of valacyclovir or its salts having specific sizes are used in embodiments of the pharmaceutical compositions of the present invention. These specific particle sizes have a beneficial influence on content uniformity and the release profile of the compositions.

In an embodiment of the present invention includes pharmaceutical compositions of valacyclovir, wherein D₉₀ of valacyclovir is in the range of about 350 μm to about 500 μm.

In an embodiment according to the present invention the pharmaceutical compositions may be formed into solid dosage forms such as tablets, capsules, granules, slugs, etc.

When formulating solid dosage forms by dry granulation or by direct compression the choice of excipients such as fillers, binders, disintegrants, lubricants, etc is important. The excipients must fulfill certain requirements: good binding functionality and powder flowability; a well-designed particle size distribution providing favorable mixing conditions; and compatibility with other excipients or drugs and the ability to carry high amounts of active ingredient. An understanding of the physico-chemical properties of these excipients is useful for their proper use.

The present invention further contains other pharmaceutically acceptable excipients, which include but are not limited to diluents or fillers, binders, glidants, lubricants, colorants, flavors, and solvents.

### Diluents or Fillers:

Various useful fillers or diluents include but are not limited to starches, lactose, mannitol, cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches included but not limited to maize starch, potato starch, rice starch, wheat starch, pregelatinized starch (Commercially available as PCS PC10 from Signet Chemical Corporation) and Starch 1500, Starch 1500 LM grade (low moisture content grade) from Colorcon, fully pregelatinized starch (Commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, and tribasic calcium phosphate.

In an embodiment the invention includes concentrations of diluents in the formulation ranging up to about 50% w/w by weight of the total composition.

### Binders:

Various useful binders include but are not limited to hydroxypropyl cellulose (Klucel™-LF), hydroxypropyl methylcellulose or hypromellose (Methocel™), polyvinylpyrrolidone or povidone (PVP-K25, PVP-K29, PVP-K30, PVP-K90), plasdone S 630 (copovidone), powdered acacia, gelatin, guar gum, carbomer (e.g. carbopol), methylcellulose, polymethacrylates, and starch.

In an embodiment the invention includes concentrations of binders in the formulation ranging up to about 25% w/w by weight of the total composition.

### Disintegrants:

Various useful disintegrants include but are not limited to carmellose calcium (Gotoku Yakuhin Co., Ltd.), carboxy methylstarch sodium (Matsutani Kagaku Co., Ltd., Kimura Sangyo Co., Ltd., etc.), croscarmellose sodium (FMC-Asahi Chemical Industry Co., Ltd.), crospovidone, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcellulose. Examples of low-substituted hydroxypropyl cellulose include but are not limited to low-substituted hydroxypropyl cellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starch.

### Glidants/Antisticking Agents:

Various glidants or antisticking agents can be used, including but not limited to talc, silica derivatives, colloidal silicon dioxide and the like, or mixtures thereof.

### Lubricants:

Various lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, zinc stearate, castor oils, and waxes

### Colourants:

Various useful colourants include but are not limited to Food Yellow No. 5, Food Red No. 2, Food Blue No. 2, and the like, food lake colorants, and iron oxides.

### Flavors:

The flavoring agents, which can be used in this present invention, include but are but not limited to those of natural or synthetic or semi-synthetic origin like menthol, fruit flavors, citrus oils, peppermint oil, spearmint oil, and oil of wintergreen (Methyl salicylate).

### Film-Forming Agents:

Various useful film-forming agents include but not limited to: cellulose derivatives such as soluble alkyl- or hydroalkyl-cellulose derivatives such as methyl cellulose, hydroxymethyl cellulose, hydroxyethyl cellulose, hydroxypropyl cellulose, hydroxymethyethyl cellulose, hydroxypropyl methylcellulose, sodium carboxymethyl cellulose, etc.; acidic cellulose derivatives such as cellulose acetate phthalate, cellulose acetate trimellitate and methylhydroxypropylcellulose phthalate, polyvinyl acetate phthalate, etc.; insoluble cellulose derivative such as ethyl cellulose and the like; dextrins; starches and starch derivatives; polymers based on carbohydrates and derivatives thereof; natural gums such as gum Arabic, xanthans, alginates; polyacrylic acid; polyvinyl alcohol; polyvinyl acetate; polyvinylpyrrolidone; polymethacrylates and derivatives thereof (Eudragit™); chitosan and derivatives thereof; shellac and derivatives thereof; waxes; and fat substances.

If desired, the films may contain additional adjuvants for coating processes such as plasticizers, polishing agents, colorants, pigments, antifoam agents, opacifiers, antisticking agents, and the like.

### Plasticizers:

Various plasticizers include but are not limited to materials such as castor oil, diacetylated monoglycerides, dibutyl sebacate, diethyl phthalate, glycerin, polyethylene glycol, propylene glycol, triacetin, and triethyl citrate. Also, mixtures of plasticizers may be utilized. The type of plasticizer depends upon the type of coating agent. A plasticizer is normally present in an amount ranging from about 5% (w/w) to about 30 (w/w), based on a total weight of the film coating.

An opacifier like titanium dioxide may also be present in an amount ranging from about 10% (w/w) to about 20% (w/w), based on a total weight of the coating. When coloured tablets are desired then the colour is normally applied in the coating. Consequently, colouring agents and pigments may be present in the film coating. Various colouring agents include but are not limited to iron oxides, which can be red, yellow, black or blends thereof.

Anti-adhesives are frequently used in film coating processes to avoid sticking effects during film formation and drying. A typical antiadhesive for this purpose is talc. The anti-adhesive such as talc can be present in the film coating in an amount of about 5% (w/w) to 15% (w/w), based upon the total weight of the coating.

Suitable polishing agents include polyethylene glycols of various molecular weights and mixtures thereof, talc, surfactants (e.g. glycerol monostearate and poloxamers), fatty alcohols (e.g., stearyl alcohol, cetyl alcohol, lauryl alcohol and myristyl alcohol) and waxes (e.g., carnauba wax, candelilla wax and white wax). In an embodiment, polyethylene glycols having molecular weights of 3,000-20,000 are employed.

In addition to the above coating ingredients, sometimes readymix coating materials can conveniently be used, such as OPADRY™ products (supplied by Colorcon), for example Opadry Blue 13B50579. Similar products are available from other sources. These products require only mixing with a liquid before use.

### Solvents:

Various solvents that can be used in the processes for preparing pharmaceutical compositions of the present invention include but are not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethyl sulphoxide, dimethylformamide, tetrahydrofuran, and mixtures thereof.

Equipment suitable for processing the pharmaceutical compositions of the present invention include mechanical sifters, blenders, roller compactors, compression machines, rotating bowls or coating pans, etc.

### Process for Preparing Pharmaceutical Compositions:

The present invention further relates to processes for manufacturing pharmaceutical compositions of the present invention, wherein an embodiment of a process comprises:

a) Sifting active ingredient;

b) Sifting excipients;

c) Mixing sifted materials;

d) Lubricating the blend of step c) by mixing with a sifted lubricant;

e) Filling the lubricated blend into empty hard gelatin capsule shells, filling into sachets, or compressing into tablets using appropriate tooling; and

f) Optionally, coating the dosage form with coating solution or dispersion.

The tablets prepared as above can be tested for physical parameters such as weight variation, hardness, disintegration test, friability etc. Several devices are commonly used to test tablet hardness such as a Monsanto tester, Strong-Cobb tester, Pfizer tester, Erweka tester, Schleuniger tester, etc. Friability is determined by Roche friabilator for 100 revolutions at 25 rpm. Disintegration testing for tablets is performed in a USP tablet disintegration tester wherein a tablet is placed in a basket, which moves upward and downward in a 1 L beaker of dissolution solution at 37° C.

The tablets prepared as above can be subjected to an in vitro dissolution evaluation according to Test 711 “Dissolution” in United States Pharmacopeia 24, United States Pharmacopeial Convention, Inc, Rockville, Md., 2000, (“USP”) to determine the rate at which the active substance is released from the dosage forms, and content of active substance can be determined in solutions using techniques such as high performance liquid chromatography. The pharmaceutical dosage forms of the present invention are intended for oral administration to a patient in need thereof.

In an embodiment the invention includes the use of packaging materials such as containers and lids of high-density polyethylene (HDPE), low-density polyethylene (LDPE), polypropylene and/or glass, and blisters or strips composed of aluminium and high-density polypropylene, polyvinyl chloride, polyvinylidene dichloride, etc.

In determining bioequivalence, for example between two products such as a commercially available product and a proposed product, pharmacokinetic studies are conducted whereby each of the preparations is administered to subjects and plasma concentrations of drug are measured at intervals after dosing. For example, a suitable bioequivalence study can be an open label, balanced randomized two treatment two sequence, two period and single dose crossover comparative bioavailability study to volunteer subjects. Serum plasma samples are obtained at regular intervals and assayed for parent drug (or occasionally metabolite) concentrations. For a pharmacokinetic comparison, the plasma concentration data are used to assess key pharmacokinetic parameters such as area under the plasma concentration-time curve (AUC), peak plasma concentration (Cₘₐₓ), and the elapsed time to peak plasma concentration (Tₘₐₓ).

In an embodiment the invention includes pharmaceutical formulations comprising valacyclovir or its salts or its derivatives producing plasma Cₘₐₓ values ranging from about 130 ng/mL to about 210 ng/mL after administration of a 1000 mg valacyclovir single dose to healthy humans in a fasting condition.

In an embodiment the invention includes pharmaceutical formulations comprising valacyclovir or its salts or its derivatives producing plasma AUC₀₋ₜ values ranging from about 240 ng-hour/mL to about 390 ng-hour/mL after administration of a 1000 mg valacyclovir single dose to healthy humans in a fasting condition.

In an embodiment the invention includes pharmaceutical formulations comprising valacyclovir or its salts or its derivatives producing plasma AUC_{0-∞} values ranging from about 250 ng-hour/mL to about 400 ng-hour/mL after administration of a 1000 mg valacyclovir single dose to healthy humans in a fasting condition.

In an embodiment the invention includes pharmaceutical formulations comprising valacyclovir or its salts or its derivatives producing plasma Cₘₐₓ values ranging from about 280 ng/mL to about 440 ng/mL after administration of a 1000 mg valacyclovir single dose to healthy humans in a fed condition.

In an embodiment the invention includes pharmaceutical formulations comprising valacyclovir or its salts or its derivatives producing plasma AUC_{0-∞}T values ranging from about 450 ng-hour/mL to about 720 ng-hour/mL after administration of a 1000 mg valacyclovir single dose to healthy humans in a fed condition.

In an embodiment the invention includes pharmaceutical formulations comprising valacyclovir or its salts or its derivatives producing plasma AUC_{0-∞} values ranging from about 460 ng-hour/mL to about 730 ng-hour/mL after administration of a 1000 mg valacyclovir single dose to healthy humans in a fed condition.

Certain specific aspects and embodiments of the invention will be further described in the following examples, which are provided only for purposes of illustration and are not intended to limit the scope of the invention in any manner.

### EXAMPLE 1

Composition of valacyclovir hydrochloride tablets (1000 mg valacyclovir).

| Ingredient | mg/Tablet |
|---|---|
| Valacyclovir hydrochloride | 1112.45 |
| Starch pregelatinized (starch 1500) | 209.55 |
| Hypromellose 15 Cps | 60 |
| Magnesium stearate | 18 |

Coating

| | |
|---|---|
| Opadry Blue 13B50579* | 42 |
| Water‡ | 308 |

‡Evaporates during processing.
*Opadry Blue 13B50579 contains hypromellose 6 cps, FDC blue #2/Indigo carmine aluminium lake, titanium dioxide, polyethylene glycol 400, polysorbate 80, and carnauba wax.

Valacyclovir hydrochloride used in this example had the following particle size distribution when tested by a sieve analysis method:

(a) Not less than 90% of particles of valacyclovir hydrochloride passed through a ASTM #40 mesh (425 microns) sieve.

(b) Not less than 20% of particles of valacyclovir hydrochloride were retained on a ASTM #60 mesh (250 microns) sieve.

Manufacturing process:

1) Valacyclovir hydrochloride was sifted through a #30 mesh sieve.

2) Starch pregelatinized and Hypromellose 15 cps was sifted through a #40 mesh sieve.

3) Sifted materials of step 1 and step 2 were mixed in a double cone blender for 15 minutes.

4) Magnesium stearate was sifted through a #60 mesh sieve.

5) Sifted magnesium stearate was added to the blend of step 3) and mixed for 5 minutes.

6) The lubricated blend of step 5) was compressed into tablet using 23×9.5 mm capsule shaped punches to an average weight of 1400 mg, containing 1000 mg of valacyclovir.

7) Coating suspension: Opadry Blue was dispersed in water and stirred for about 45 minutes.

8) The core tablets of step 6 were coated with coating suspension of step 7) in a perforated coating pan to get a weight buildup of about 2-3% w/w.

Physical parameters of the lubricated blend of step 5 are shown in Table 1.

TABLE 1

| Parameter | Value |
|---|---|
| Water by KF (%) | 8.4 |
| Bulk density (g/ml) | 0.65 |
| Tapped density (g/ml) | 0.92 |

Particle size

| Sieve Mesh # | Cumulative % Retained |
|---|---|
| 30 | 0.1 |
| 40 | 1 |
| 60 | 25 |
| 80 | 40 |
| 100 | 46 |
| <100 | 100 |

Physical parameters (average) of core tablets from step 6 are given in Table 2.

TABLE 2

| Parameter | Value |
|---|---|
| Hardness, Kilopond (kp) | 24 |
| Disintegration time (minutes) | 14.5 |
| Friability (%) | 0.082 |

The pharmacokinetic parameters Cₘₐₓ (maximum concentration of drug in the plasma), AUC₀₋ₜ (area under the curve from time 0 to time t=96 hours), and AUC_{0-∞} (area under the curve from time 0 to time infinity) have been determined for the above preparation. The T/R ratios are given in Table 3 and individual mean results are given in Table 4, where T represents the “Test” product of Example 1 and R represents the “Reference” product, VALTREX® 1000 mg.

Study design: Open label, balanced, randomized, two treatment, two sequence, two-period, single dose, crossover comparative bioavailability study under fasting and fed conditions.

Number of subjects: 42 healthy adult human subjects.

TABLE 3

| Conditions | Cₘₐₓ (T/R) % | AUC₀₋ₜ (T/R) % | AUC_{0-∞} (T/R) % |
|---|---|---|---|
| Fasting | 89.21 | 98.44 | 98.5 |
| Fed | 89.76 | 101.73 | 101.49 |

TABLE 4

| | |
|---|---|
| Fasting | Fed |

| Parameter | Test | Reference | Test | Reference |
|---|---|---|---|---|
| AUC₀₋ₜ | 304.505 | 309.317 | 579.863 | 569.99 |
| (ng · hour/ml) | | | | |
| AUC_{0-∞} | 311.255 | 315.999 | 587.525 | 578.91 |
| (ng · hour/ml) | | | | |
| Cₘₐₓ (ng/ml) | 147.042 | 164.821 | 315.263 | 351.219 |

### EXAMPLE 1A

Composition of valacyclovir hydrochloride tablets (500 mg valacyclovir).

A lubricated blend prepared with the same component ratios as in preceding Example 1 was compressed into tablets using 18.4×7.5 mm capsule shaped punches, to an average weight of 700 mg, and further coated as in Example 1.

Physical parameters (average) of core tablets are given in Table 5.

TABLE 5

| Parameter | Value |
|---|---|
| Hardness (kp) | 19 |
| Disintegration time (minutes) | 12 |
| Friability (%) | 0.092 |

### EXAMPLE 2

Comparative stability for compositions prepared according to Examples 1 and VALTREX® 1000 mg.

Tablets prepared according to Example 1 and VALTREX® 1000 mg were stored under the accelerated stability conditions 40° C. and 75% relative humidity (RH) for three months, and samples were analyzed at intervals for related impurities and purity.

### EXAMPLE 2A

Tablets were packaged in blisters made of PVC film, coated with PVDC 60 gsm (grams per square meter) and backed with aluminum foil. The data from tested parameters are given below in Table 6.

TABLE 6

| Sample | Time | Purity | Acyclovir | G | U | TI |
|---|---|---|---|---|---|---|
| Example 2A | Initial | 101 | 0.15 | 0.02 | 0.03 | 0.15 |
| | 1 Month | 99.6 | 0.16 | 0.03 | 0.03 | 0.16 |
| | 2 Months | 99.9 | 0.19 | 0.03 | 0.04 | 0.18 |
| | 3 Months | 100.8 | 0.18 | 0.03 | 0.05 | 0.19 |
| VALTREX | Initial | 99.9 | 1.05 | 0.007 | 0.09 | 1.17 |
| 1000 mg | 1 Month | 99.6 | 1.05 | 0.007 | 0.11 | 1.17 |
| | 2 Months | 99.8 | 1.01 | 0.007 | 0.11 | 1.15 |
| | 3 Months | 99.6 | 0.99 | ND | 0.09 | 1.16 |

Abbreviations: G = Gaunine impurity; U = Unknown; TI = Total impurities.
“Purity” is percent of theoretical valacyclovir concentration. Values for impurities are percentages of the contained valacyclovir.

Tablets prepared according to Example 1A were also packed in alternative packages and stored under accelerated stability conditions (40° C./75% RH) for six months, and samples were analyzed at intervals for impurities, dissolution and purity.

### EXAMPLE 2B

Tablets were packed in closed 40 ml HDPE containers having closures of 33 mm child resistant plastic caps.

### EXAMPLE 2C

Tablets were packed in closed 500 ml HDPE containers having closures of 53 mm ribbed smooth plastic caps.

The data from tested parameters are given below in Table 7.

TABLE 7

| Sample | Time | Purity | Acyclovir | G | U | TI |
|---|---|---|---|---|---|---|
| Example 2B | Initial | 101 | 0.51 | 0.02 | 0.03 | 0.14 |
| | 1 Month | 99.1 | 0.6 | 0.03 | 0.03 | 0.16 |
| | 2 Months | 99.5 | 0.56 | 0.03 | 0.03 | 0.18 |
| | 3 Months | 99.2 | 0.59 | 0.03 | 0.05 | 0.2 |
| | 6 Months | 100.8 | 0.59 | 0.03 | 0.03 | 0.19 |
| Example 2C | Initial | 101 | 0.5 | 0.02 | 0.03 | 0.14 |
| | 1 Month | 99.3 | 0.59 | 0.03 | 0.03 | 0.16 |
| | 2 Months | 100 | 0.57 | 0.03 | 0.03 | 0.19 |
| | 3 Months | 100 | 0.6 | 0.03 | 0.05 | 0.19 |
| | 6 Months | 100 | 0.55 | 0.03 | 0.03 | 0.16 |

Abbreviations: G = Gaunine impurity; U = Unknown; TI = Total impurities.
“Purity” is percent of theoretical valacyclovir concentration. Values for impurities are percentages of the contained valacyclovir.

### EXAMPLE 3

Stability studies for compositions prepared according to Example 1A in alternate packages.

Tablets prepared according to Example 1A were packaged in three different packages and stored under accelerated stability conditions (40° C./75% RH) for six months, and samples were analyzed at intervals for related impurities, dissolution and purity.

### EXAMPLE 3A

Tablets were packaged in closed 40 ml HDPE containers having closures of 33 mm child resistant plastic caps.

### EXAMPLE 3B

Tablets were packaged in closed 500 ml HDPE containers having closures of 53 mm ribbed smooth plastic caps.

### EXAMPLE 3C

Tablets were packaged in clear blisters formed from PVC film coated with PVDC 60 gsm and backed with aluminum foil.

The data from tested parameters are given below in Table 8.

TABLE 8

| Sample | Time | Purity | Acyclovir | G | U | TI |
|---|---|---|---|---|---|---|
| Example 3A | Initial | 98.4 | 0.5 | 0.02 | 0.03 | 0.15 |
| | 1 Month | 99.3 | 0.58 | 0.03 | 0.03 | 0.15 |
| | 2 Months | 99.1 | 0.56 | 0.03 | 0.04 | 0.19 |
| | 3 Months | 100 | 0.6 | 0.03 | 0.05 | 0.19 |
| | 6 Months | 101 | 0.58 | 0.03 | 0.03 | 0.16 |
| Example 3B | Initial | 98.4 | 0.48 | 0.02 | 0.03 | 0.14 |
| | 1 Month | 99.5 | 0.58 | 0.03 | 0.03 | 0.16 |
| | 2 Months | 101.5 | 0.58 | 0.03 | 0.04 | 0.2 |
| | 3 Months | 101.7 | 0.61 | 0.03 | 0.05 | 0.18 |
| | 6 Months | 98.5 | 0.8 | 0.03 | 0.02 | 0.15 |
| Example 3C | Initial | 98.4 | 0.5 | 0.02 | 0.03 | 0.15 |
| | 1 Month | 99.1 | 0.57 | 0.02 | 0.03 | 0.16 |
| | 2 Months | 100.3 | 0.56 | 0.03 | 0.03 | 0.19 |
| | 3 Months | 100.1 | 0.58 | 0.03 | 0.05 | 0.18 |
| | 6 Months | 98 | 0.59 | 0.03 | 0.03 | 0.17 |

Abbreviations: G = Gaunine impurity; U = Unknown; TI = Total impurities.
“Purity” is percent of theoretical valacyclovir concentration. Values for impurities are percentages of the contained valacyclovir.

### EXAMPLE 4

Composition of valacyclovir hydrochloride tablets (1000 mg valacyclovir) with other diluents and binders.

| Ingredient | mg/Tablet |
|---|---|
| Valacyclovir hydrochloride | 1112.45 |
| Microcrystalline cellulose | 229.55 |
| Crospovidone | 14 |
| Povidone K 90 | 30 |
| Magnesium stearate | 14 |

Coating

| | |
|---|---|
| Opadry Blue 13B50579 | 42 |
| Water ‡ | 308 |

‡ Evaporates during processing.

Manufacturing process: similar to that of Example 1.

Physical Parameters of Core Tablets:

| Parameter | Value |
|---|---|
| Hardness (kp) | 17-20 |
| Disintegration time (minutes) | 25 |
| Friability (%) | 0.11% |

### EXAMPLE 5

Composition of valacyclovir hydrochloride tablets (1000 mg valacyclovir) with mannitol and copovidone.

| Ingredient | mg/Tablet |
|---|---|
| Valacyclovir hydrochloride | 1112.45 |
| Mannitol | 209.55 |
| Copovidone (Plasdone ™ S 630) | 60 |
| Magnesium stearate | 18 |

Coating

| | |
|---|---|
| Opadry Blue 13B50579 | 42 |
| Water ‡ | 308 |

‡ Evaporates during processing.

Manufacturing process: similar to that of Example 1.

### EXAMPLE 6

Composition of valacyclovir hydrochloride tablets (1000 mg valacyclovir) with microcrystalline cellulose and copovidone.

| Ingredient | mg/Tablet |
|---|---|
| Valacyclovir hydrochloride | 1112.45 |
| Microcrystalline cellulose | 209.55 |
| Copovidone (Plasdone S 630) | 60 |
| Magnesium stearate | 18 |

Coating

| | |
|---|---|
| Opadry Blue 13B50579 | 42 |
| Water ‡ | 308 |

‡ Evaporates during processing.

Manufacturing process: similar to that of Example 1.

### EXAMPLE 7

Composition of valacyclovir hydrochloride tablets (1000 mg valacyclovir) with dicalcium phosphate and copovidone.

| Ingredient | mg/Tablet |
|---|---|
| Valacyclovir hydrochloride | 1112.45 |
| Dicalcium phosphate | 209.55 |
| Copovidone (Plasdone S 630) | 60 |
| Magnesium stearate | 18 |

Coating

| | |
|---|---|
| Opadry Blue 13B50579 | 42 |
| Water ‡ | 308 |

‡ Evaporates during processing.

Manufacturing process: similar to that of Example 1.

### EXAMPLE 8

Composition of valacyclovir hydrochloride tablets (1000 mg valacyclovir) with starch pregelatinized and povidone.

| Ingredient | mg/Tablet |
|---|---|
| Valacyclovir hydrochloride | 1112.45 |
| Starch pregelatinized | 209.55 |
| Povidone K 90 | 60 |
| Magnesium stearate | 18 |

Coating

| | |
|---|---|
| Opadry Blue 13B50579 | 42 |
| Water ‡ | 308 |

‡ Evaporates during processing.

Manufacturing process: similar to that of Example 1.

### EXAMPLE 9

Composition of valacyclovir hydrochloride tablets (1000 mg valacyclovir) with starch pregelatinized and hypromellose.

| Ingredient | mg/Tablet |
|---|---|
| Valacyclovir hydrochloride | 1112.45 |
| Starch pregelatinized | 169.55 |
| Hypromellose 15 Cps | 100 |
| Magnesium stearate | 18 |

Coating

| | |
|---|---|
| Opadry Blue 13B50579 | 42 |
| Water ‡ | 308 |

‡ Evaporates during processing.

Manufacturing process: similar to that of Example 1.

### EXAMPLE 10

Composition of valacyclovir hydrochloride tablets (1000 mg valacyclovir) with starch pregelatinized and Methocel K 100 M CR

| Ingredient | mg/Tablet |
|---|---|
| Valacyclovir hydrochloride | 1112.45 |
| Starch pregelatinized | 244.55 |
| Methocel K 100 M CR | 25 |
| Magnesium stearate | 18 |

Coating

| | |
|---|---|
| Opadry Blue 13B50579 | 42 |
| Water ‡ | 308 |

‡ Evaporates during processing.

Manufacturing process: similar to that of Example 1.

### EXAMPLE 11

Composition of valacyclovir hydrochloride tablets (1000 mg valacyclovir) by dry granulation.

| Ingredient | mg/Tablet |
|---|---|
| Valacyclovir hydrochloride | 1217 |
| Avicel PH 101 | 61 |
| Polyvinylpyrrolidone PVP K-90 | 40 |
| Crospovidone | 14 |
| Avicel PH 102 | 25 |
| Magnesium stearate | 18 |

Manufacturing process:

1) Valacyclovir hydrochloride was sifted through a ASTM #30 mesh sieve. Avicel was sifted through a ASTM #60 mesh sieve and polyvinylpyrrolidone was sifted through a ASTM #40 mesh sieve.

2) Step 1 ingredients were blended uniformly in a double cone blender for about 15 minutes.

3) Step 2 blend was compacted into flakes in a roller compactor three times.

4) Step 3 flakes were passed through a ASTM #20 mesh sieve and the retained fraction was milled in a comminuting mill using a 1.5 mm screen, then combined with the sifted fraction.

5) Step 4 flakes were sifted through a ASTM #20 mesh sieve and crospovidone previously sifted through a ASTM #40 mesh sieve was added to the and blended uniformly for 5 minutes.

6) Magnesium stearate was passed through a ASTM #60 mesh sieve and added to the step 5) blend.

7) Step 6 was compressed into 1400 mg tablets using 23×9.5 mm punches.

### EXAMPLE 12

Composition of valacyclovir hydrochloride tablets (1000 mg Valacyclovir) by dry granulation.

| Ingredient | mg/Tablet |
|---|---|
| Valacyclovir hydrochloride | 1217 |
| Pregelatinised starch | 151 |
| Sodium starch glycolate | 14 |
| Magnesium stearate | 18 |

Manufacturing process:

1) Valacyclovir hydrochloride was sifted through ASTM #30 mesh sieve. Pregelatinised starch was sifted through a ASTM #60 mesh sieve and both ingredients were blended uniformly for 15 minutes in a double cone blender.

2) Step 1 blend was compacted using a roller compactor.

3) The material (flakes) was separated from fines by passing through a ASTM #20 mesh sieve.

4) Material of step 3 was passed through a ASTM #20 mesh sieve, then was again compacted and sifted through a ASTM #20 mesh sieve.

5) All the compacted materials and granules retained on the sieve were milled through a 1.5 mm screen in a comminuting mill and sifted through a ASTM #20 mesh sieve.

6) Sodium starch glycolate was sifted through a ASTM #60 mesh sieve, added to step 5 and blended uniformly for 5 minutes.

7) Magnesium stearate was passed through a ASTM #60 mesh sieve and blended with the step 6 blend.

8) Step 7 was compressed into 1400 mg tablets using 23×9.5 mm punches.

### EXAMPLE 13

Comparative dissolution profiles for Examples 1, 4, 5, 9, 10 and 1A, and VALTREX® 1000 mg and 500 mg caplets.

Media: 900 ml of 0.1N HCl.

Apparatus: USP Apparatus II.

Speed: 50 rpm.

Results are tabulated in Tables 9 and 10, where values are percent of contained valacyclovir released.

TABLE 9

| Time | VALTREX ® | Example | Example | Example | Example | Example |
|---|---|---|---|---|---|---|
| (Minutes) | 1000 mg | 1 | 4 | 5 | 9 | 10 |
| 15 | 33 | 68 | 96 | 102 | 61 | 51 |
| 30 | 66 | 99 | 102 | 104 | 94 | 86 |
| 45 | 87 | 102 | 102 | 105 | 100 | 98 |
| 60 | 97 | 102 | 102 | 106 | 101 | 101 |

TABLE 10

| Time | VALTREX ® | Example |
|---|---|---|
| (minutes) | 500 mg | 1A |
| 15 | 71 | 79 |
| 30 | 93 | 98 |
| 45 | 101 | 100 |
| 60 | 102 | 100 |

We claim:

1. A process to prepare a solid pharmaceutical composition comprising valacyclovir or a salt thereof, wherein process steps do not include wet granulation.

2. The process according to claim 1, comprising particle formation by dry granulation.

3. The process according to claim 1, comprising tablet formation by direct compression of a powder blend.

4. A pharmaceutical composition prepared by the process of claim 1, wherein a guanine impurity does not exceed about 0.25 percent by weight of the valacyclovir or salt concentration.

5. A pharmaceutical composition prepared by the process of claim 1, wherein an acyclovir impurity does not exceed about 1 percent by weight of the valacyclovir or salt concentration.

6. A pharmaceutical composition prepared by the process of claim 1, wherein a total impurity content does not exceed about 2 percent by weight of the valacyclovir or salt concentration.

7. A pharmaceutical composition prepared by the process of claim 1, producing: Cₘₐₓ values ranging from about 280 ng/mL to about 440 ng/mL; AUC₀₋ₜ values ranging from about 450 ng-hour/mL to about 720 ng-hour/mL; and AUC_{0-∞} values ranging from about 460 ng-hour/mL to about 730 ng-hour/mL; following administration of a 1000 mg valacyclovir single dose to healthy humans in a fed condition.

8. A pharmaceutical composition prepared by the process of claim 1, producing: Cₘₐₓ values ranging from about 130 ng/mL to about 210 ng/mL; AUC₀₋ₜ values ranging from about 240 ng-hour/mL to about 390 ng-hour/mL; and AUC_{0-∞} values ranging from about 250 ng-hour/mL to about 400 ng-hour/mL; following administration of a 1000 mg valacyclovir single dose to healthy humans in a fasting condition.

9. A pharmaceutical composition prepared by the process of claim 1, using valacyclovir or a salt thereof having a particle size distribution wherein D₉₀ is about 350 μm to about 500 μm.

10. A process to prepare a solid pharmaceutical composition comprising a salt of valacyclovir, comprising particle formation by dry granulation, wherein process steps do not include wet granulation.

11. A pharmaceutical composition prepared by the process of claim 10, wherein a guanine impurity does not exceed about 0.25 percent by weight of the valacyclovir salt concentration.

12. A pharmaceutical composition prepared by the process of claim 10, wherein an acyclovir impurity does not exceed about 1 percent by weight of the valacyclovir salt concentration.

13. A pharmaceutical composition prepared by the process of claim 10, wherein a total impurity content does not exceed about 2 percent by weight of the valacyclovir salt concentration.

14. A process to prepare a solid pharmaceutical composition comprising a salt of valacyclovir, comprising particle formation by direct compression of a powder blend, wherein process steps do not include wet granulation.

15. A pharmaceutical composition prepared by the process of claim 14, wherein a guanine impurity does not exceed about 0.25 percent by weight of the valacyclovir salt concentration.

16. A pharmaceutical composition prepared by the process of claim 14, wherein an acyclovir impurity does not exceed about 1 percent by weight of the valacyclovir salt concentration.

17. A pharmaceutical composition prepared by the process of claim 14, wherein a total impurity content does not exceed about 2 percent by weight of the valacyclovir salt concentration.
